(19) United States
(12) Patent Application Publication
(10) Pub. No.: US 2014/0180104 A1
(43) Pub. Date: Jun. 26, 2014

(54) ULTRASONIC PIERCING NEEDLE

(71) Applicant: HAKKO CO., LTD., Chikuma-shi (JP)

(72) Inventors: Masaru Maruyama, Chikuma-shi (JP); Shiro Kitagawa, Tokyo (JP); Masayoshi Komatsu, Sendai-shi (JP)

(73) Assignee: HAKKO CO., LTD., Chikuma-shi (JP)

(21) Appl. No.: 14/194,513

(22) Filed: Feb. 28, 2014

Related U.S. Application Data
(63) Continuation of application No. 11/989,450, filed on Jan. 25, 2008.

(51) Int. Cl. A61B 8/08

(52) U.S. Cl. CPC A61B 8/0841
USPC 600/439

(57) ABSTRACT

An ultrasonic puncture needle for conducting puncturing while observing a puncturing condition by an ultrasonic image, includes a needle tube main body including an edge at its tip end, and a plurality of corner cube mirrors provided at a predetermined position of the needle tube main body, wherein the plurality of corner cube mirrors are provided in parallel with a center axis of the needle tube main body.

The present application is a Continuation Application of U.S. patent application Ser. No. 11/989,450 filed on Jan. 25, 2008, which is based on International Application No. PCT/JP2005/013603 filed on Jul. 25, 2005.

### BACKGROUND OF THE INVENTION

1. Field of the Invention

The present invention relates to a piercing (i.e., puncture) needle for a medical application, and more particularly, to a puncture needle used under displaying ultrasonic image.

2. Related Art

In the medical field, a puncture needle (biopsy needle, insulated electrode needle) is used, for example, for gathering a part of a tissue or a cell of a human organism is gathered for the purpose of diagnostic confirmation of a lesion, or for precisely finding a location of a nerve. For this case, an ultrasonic diagnostic equipment is used from a requirement of precisely recognizing a location of the puncture needle, particularly a location of tip end thereof. For example, in a case of using a biopsy needle, needle puncturing is conducted while observing an ultrasonic echo from the tip end of the puncture needle by displaying a pictorial image, to precisely finding as to whether a tissue collecting part of the tip end of the puncture needle (a needle point or a notch) reaches the lesion. Further, this biopsy needle often has a dual structure comprising an inner tube and an outer tube.

On the other hand, another puncture needle called “an insulated electrode block needle” in general has a single tube structure (single-needle), in that an electrical insulation coating is provided on a surface of the needle tube except an edge surface thereof The insulated electrode block needle is generally used for searching the nerve by using a muscle contraction caused by an electric stimulation, in order to conduct a nerve block by dosing anesthetics or painkiller to the nerve. In recent years, a manual technique to puncture the needle while observing the ultrasonic image together with energization by a conventional nerve stimulator (puncturing under irradiation of the ultrasonic wave and confirmation by the nerve stimulator) has been widely used in an approach of the nerve search.

The ultrasonic image is an image obtained by irradiating an ultrasonic wave from an ultrasonic probe (ultrasonic transducer) as a transmitting and receiving device of the ultrasonic wave to the puncture needle via a medium, receiving a reflected wave (ultrasonic echo) from the puncture needle at the ultrasonic probe, and conducting an image processing on the reflected wave to display an image on a display unit.

As the conventional puncture needles to be used while displaying the ultrasonic image, for example, a puncture needle provided with a protrusion, a V-groove or the like at its outer periphery surface of the tip end (for example, see patent document 1), a puncture needle provided with a circular groove at its outer periphery surface (for example, see patent documents 2, 3), a puncture needle provided with an enhanced portion comprising a cylindrical protruding portion and a circular groove at its tip end (for example, see patent documents 4, 5) are known. Configuration of the protrusion, the groove and the like provided at the tip end of any of these puncture needles is designed for easily generating a reflection of the ultrasonic wave.

Patent document 1: Japanese Patent Laid-Open No. 3-228748

Patent document 2: Japanese Patent Laid-Open No. 11-76254

Patent document 3: Japanese Utility Model Laid-Open No. 3-73113

Patent document 4: Japanese Patent Laid-Open No. 2004-181095

Patent document 5: Japanese Patent Laid-Open No. 2003-144436

However, according to the conventional puncture needles in which the ultrasonic wave is reflected back by machining the tip end, an angle made by an irradiation path of the ultrasonic wave and an axis of the puncture needle is small. In particular, when the angle is not greater than 45°, a level of the ultrasonic echo returned to the ultrasonic probe is decreased, so that the ultrasonic image of the puncture needle becomes unclear, and as a result, it is difficult to recognize a precise location of the puncture needle.

### SUMMARY OF THE INVENTION

Accordingly, it is an exemplary object of the invention to provide a puncture needle, which can be clearly imaged, even though an angle made by an irradiation angle of a detection wave such as an ultrasonic wave of an ultrasonic probe and an axis of the puncture needle is small.

According to a feature of the present invention, so as to solve the above and other exemplary problems, an ultrasonic puncture needle for conducting puncturing while observing a puncturing condition by an ultrasonic image, comprising: a needle tube main body comprising an edge at its tip end; and a plurality of corner cube mirrors provided at a predetermined position of the needle tube main body, wherein the plurality of corner cube mirrors are provided in parallel with a center axis of the needle tube main body.

According to another feature of the present invention, an ultrasonic puncture needle, for conducting puncturing while observing a puncturing conduction by an ultrasonic image, includes a needle tube main body comprising an edge at a tip end of the needle tube main body, and a plurality of reflecting means provided on a surface of the needle tube main body in parallel with a center axis of the needle tube main body, for reflecting an incident ultrasonic wave toward an incident direction, each of the reflecting means comprising a triangular-pyramid shaped recess having an apex as a bottom point.

### EFFECTS OF THE INVENTION

According to the puncture needle of the present invention, it is possible to clearly image a needle tube main body, particularly a tip end of the needle tube main body, even though an angle made by an irradiation angle of a detection wave such as an ultrasonic wave of an ultrasonic probe and an axis of the puncture needle is small.

### BRIEF DESCRIPTION OF THE DRAWINGS

FIGS. 1A-1B shows a puncture needle in a preferred embodiment according to the present invention, wherein FIG. 1A is a perspective view thereof, and FIG. 1B is a cross sectional view thereof along A-A line in FIG. 1A;

FIGS. 2A-2B shows a corner cube mirror shown in FIG. 1, wherein FIG. 2A is an explanatory diagram showing a structure and a function of the corner cube mirror, and FIG. 2B is an explanatory diagram showing an example of application; and

FIGS. 3A-3B shows a double needle provided with the corner cube mirror, wherein FIG. 3A is a perspective view of a structure in which the corner cube mirror is provided at an edge surface of an inner needle, and FIG. 3B is a perspective view of a structure in which the corner cube mirror is provided at an inner periphery surface of an outer needle.

### REFERENCE NUMERALS

1 puncture needle
11 puncture needle main body
12 corner cube mirror
12a, 12b, 12c first to third mirror surfaces
13 edge
14 edge surface
20 ultrasonic probe
21 outer needle
22 edge surface of the inner needle
24 edge surface of the outer needle
30 double needle

### DETAILED DESCRIPTION OF THE PREFERRED EMBODIMENT

### Best Mode for Carrying out the Invention

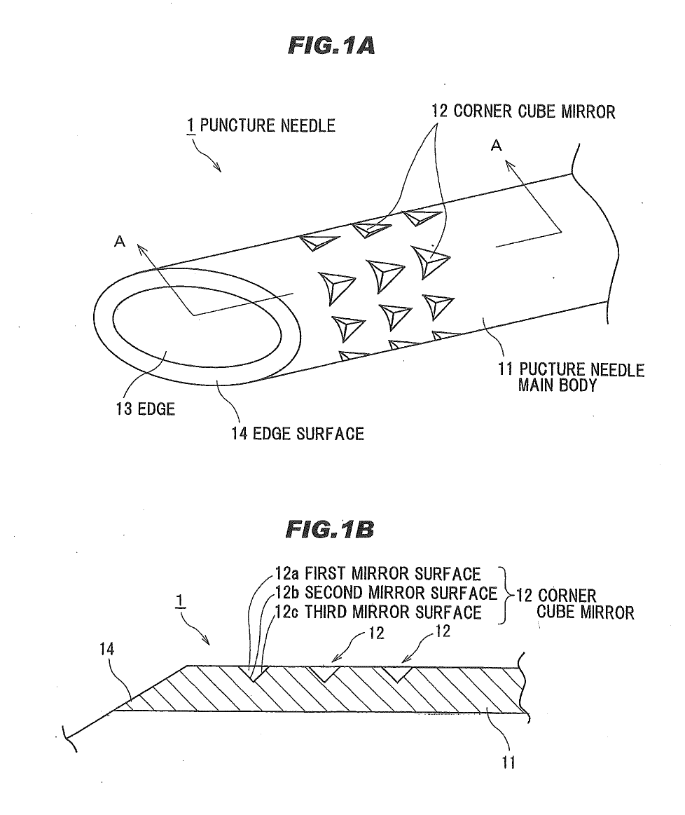

FIGS. 1A-1B shows a puncture needle in a preferred embodiment according to the present invention. In FIG. 1A is a perspective view thereof, and FIG. 1B is a cross sectional view thereof along A-A line in FIG. 1A.

An ultrasonic puncture needle (hereinafter referred as “puncture needle”) 1, which is a needle tube, comprises a puncture needle main body 11 which is a needle tube main body comprising a metal capillary made of stainless steel or the like, and a plurality of corner cube mirrors 12 provided at an outer surface (outer periphery surface) of a tip end of the puncture needle main body 11.

The puncture needle main body 11 comprises an edge 13 including an edge surface 14 made by diagonally cutting its tip portion. The corner cube mirror 12 comprises a triangular pyramid-shaped recess provided at a surface of the puncture needle main body 11 at a region in vicinity of the edge 13, and the corner cube mirrors 12 are formed along three lines with a predetermined interval in a circumferential direction of the puncture needle main body 11. For example, the corner cube mirrors 12 are disposed with an interval of 45° with respect to a center of the needle tube, and eight mirrors are formed per one line. In addition, the number of the corner cube mirrors 12 to be used and the number of lines are not limited to eight and three respectively as described above, and may be arbitrary numbers. The corner cube mirrors 12 are provided at the surface of the puncture needle main body 11, however, the present invention is not limited thereto, and may be provided at an inner periphery surface of the puncture needle main body 11 or both of them.

It is preferable to form the corner cube mirror 12 in vicinity of the edge 13, so as to clarify an imaging of the tip end. For example, the corner cube mirror 12 may be formed by electric discharge machining. Alternatively, the corner cube mirror 12 may be punched by a tool such as punches.

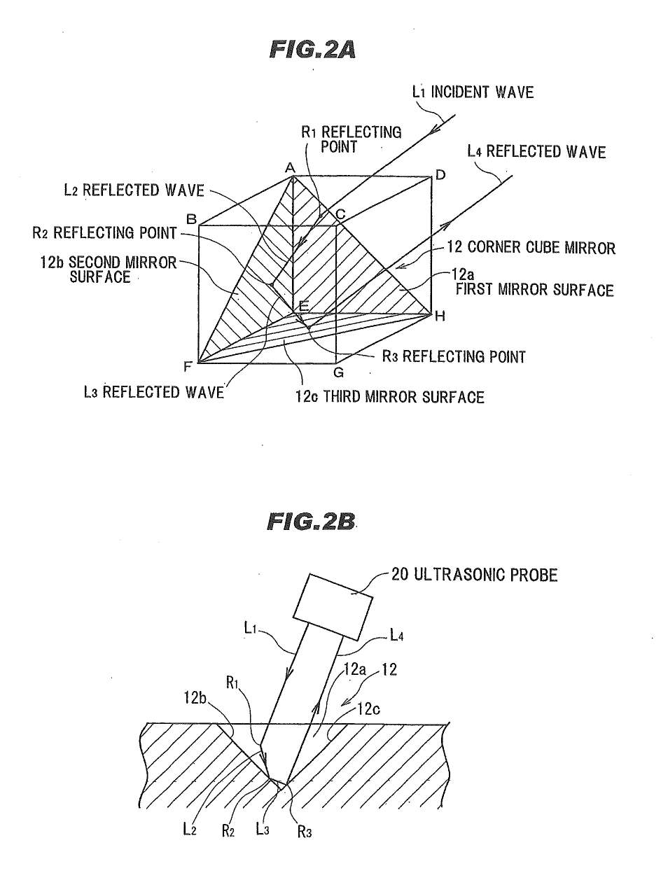

FIGS. 2A-2B shows a function of the corner cube mirror. This corner cube mirror 12 is configured by three mirror surfaces that are perpendicular to each other, and an incident wave is reflected back by the three mirror surfaces, and emitted as an output wave that is parallel to the incident wave.

As shown in FIG. 2A, three faces (ABFE, EFGH, AEHD) of a cube (ABCD-EFGH) are provided as mirror surfaces, and AFH is opened. An opening with a triangular-pyramid shape having a base point E and a top face AFH is provided as the corner cube mirror 12.

In FIG. 2A, an incident wave L₁ which is incident to the corner cube mirror 12 is reflected at a reflecting point R₁ of a first mirror surface 12a in the AEHD face to provide a reflected wave L₂, the reflected wave L₂ is reflected at a reflecting point R₂ of a second mirror surface 12b in the ABFE face to provide a reflected wave L₃, and the reflected wave L₃ is reflected at a reflecting point R₃ of a third mirror surface 12c in the EFGH face to provide an output wave L₄.

In FIG. 2B, the incident wave L₁ is emitted from an ultrasonic probe 20, and the output wave L₄ is received by the ultrasonic probe 20. According to this structure it is possible to detect the edge 13 including the edge surface 14 of the puncture needle main body 11 of the puncture needle 1 by virtue of the corner cube mirrors 12.

As described above, the incident wave and the reflected wave of the ultrasonic wave which is incident to the corner cube mirrors 12 are parallel or substantially parallel to each other. Therefore, the ultrasonic wave transmitted from the ultrasonic probe 20 is necessarily reflected at the corner cube mirror 12 to be returned to the ultrasonic probe 20, so that the reflected wave is directed to an output direction of the incident wave of the ultrasonic wave and surely returned to the ultrasonic probe 20 that is an emitting side of the ultrasonic wave, even when the angle made by the irradiation path of the ultrasonic wave and the axis of the puncture needle is small.

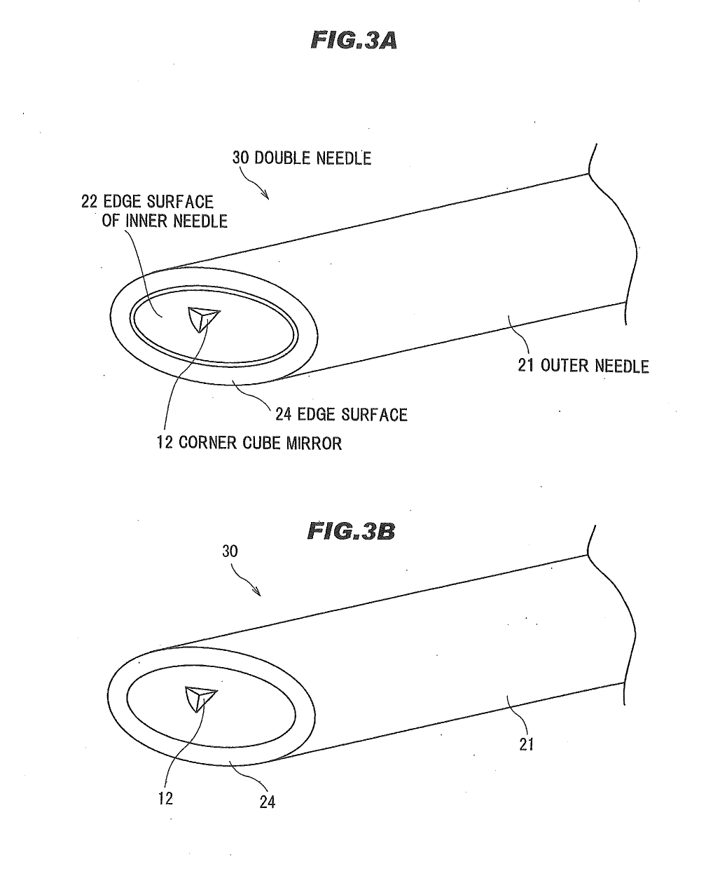

FIGS. 3A-3B shows a double needle 30 provided with the corner cube mirrors, wherein FIG. 3A is a perspective view of the double needle 30 in which an inner needle (no reference numeral) having an edge surface 22 is inserted into an outer needle 21 having an edge surface 24, and FIG. 3B is a perspective view of the outer needle 21 from which the inner needle is detached.

In FIG. 3A, the inner needle comprises the corner cube mirrors 12 provided on the edge surface 22. According to this structure, it is possible to precisely detect a tip end of the double needle 30.

In FIG. 3B, the corner cube mirror 12 is provided on an inner periphery surface of the edge surface 24 of the outer needle 21. According to this structure, it is possible to precisely detect a tip end of the outer needle 21 in the state that the inner needle is detached.

(Effect of the Preferred Embodiment)

According to this preferred embodiment, the following effects can be obtained.

(a) By providing the puncture needle with the corner cube mirror, the incident wave and the reflected wave of the ultrasonic wave in the puncture needle are made parallel or substantially parallel to each other, so that the ultrasonic wave output from the ultrasonic probe is reflected at the puncture needle and returned to the ultrasonic probe. Therefore, even though the angle made by the irradiation path of the ultrasonic wave and the axis of the puncture needle is reduced, the ultrasonic wave is reflected by the corner cube mirror and returned to the ultrasonic probe, so that it is possible to clearly image the puncture needle, particularly, the tip end thereof.

(b) By providing the corner cube mirror in vicinity of the edge, it is possible to clarify the imaging of the tip end.

(c) By providing a plurality of the corner cube mirrors at a periphery of the puncture needle, the incident wave and the reflected wave of the ultrasonic wave in the puncture needle are made parallel or substantially parallel to each other regardless of the orientation of the puncture needle, so that it is possible to clearly image the puncture needle regardless of the orientation of the puncture needle.

(d) By providing the corner cube mirrors in plural lines, it is possible to surely generate the reflected wave.

(e) Since the corner cube mirror has a good reflecting efficiency, it is possible to obtain a good reflecting property regardless of the type of the puncture needle. In particular, by providing the corner cube mirror at the outer surface of the inner needle of the double needle, it was possible to obtain an excellent reflecting property by a synergistic effect with an air layer existing between the inner needle and the outer needle. However, it was also possible to obtain the excellent reflecting property according to a structure in which the corner cube mirror is provided at an outer surface of the single tube structure and a coating is provided at the outer periphery thereof.

[Other Preferred Embodiments]

The present invention is not limited to the preferred embodiment described above, and may be modified without going beyond or transforming a technical concept of the present invention.

For example, the present invention may be applied to all puncture needles to be punctured to a target while observing a location of the needle tube punctured into the human body by the ultrasonic echo, such as a PTC (Percutaneous Transhepatic Cholangiography) needle for cholangiography, a puncture needle for an ultrasonic endoscope, and the like.

In case that a fluorine resin is coated on a part other than the edge 13 in the puncture needle provided with the corner cube mirror 12, in order to compose an insulated electrode block needle, it possible to clearly image the insulated electrode block needle, similarly to the above. By the way, since the conventional insulated electrode block needle does not comprise the corner cube mirror 12, the image was unclear due to an attenuation caused by the insulation coating.

In addition, the present invention is not limited to the use of the ultrasonic wave, and may be applied, for example, to a light such as a laser beam, electron beam, or the like. Therefore, the present invention may be applied to an application for confirming a location of an article to be detected such as needle by using the reflected wave of an electromagnetic wave by the corner cube mirror. Further, the reflecting surface of the corner cube mirror 12 is not necessarily a plane surface.

### INDUSTRIAL APPLICABILITY

The corner cube mirror formed at the outer surface of the needle tube can generate the reflected wave that is parallel or substantially parallel to the incident wave of the detection wave of the ultrasonic wave or the like, so that it is applicable to a purpose for clarifying the image of the needle tube existing in a range to which the detection wave of the ultrasonic wave or the like output from the ultrasonic probe or the like reaches, for example, a medical apparatus and a medical equipment.

What is claimed is:

1. An ultrasonic puncture needle for conducting puncturing while observing a puncturing condition by an ultrasonic image, comprising:
a needle tube main body comprising an edge at its tip end; and
a plurality of corner cube mirrors provided at a predetermined position of the needle tube main body,
wherein the plurality of corner cube mirrors are provided in parallel with a center axis of the needle tube main body.

2. The ultrasonic puncture needle, according to clam 1, wherein the plurality of corner cube mirrors are provided at an outer surface of the needle tube main body.

3. The ultrasonic puncture needle, according to claim 2, wherein the plurality of the corner cube mirrors are formed in plural lines in a longitudinal direction of the outer surface of the needle tube main body.

4. The ultrasonic puncture needle, according to claim 1, wherein the needle tube main body comprises a single tube structure, an inner needle of a double needle, or an outer needle of the double needle.

5. The ultrasonic puncture needle, according to claim 4, wherein the plurality of corner cube mirrors are provided at an inner surface of an edge of the outer needle.

6. The ultrasonic puncture needle, according to claim 1, wherein the needle tube main body is provided with a resin coating at a part other than the edge.

7. An ultrasonic puncture needle for conducting puncturing while observing a puncturing conduction by an ultrasonic image, comprising:
a needle tube main body comprising an edge at a tip end of the needle tube main body; and
a plurality of reflecting means provided on a surface of the needle tube main body in parallel with a center axis of the needle tube main body, for reflecting an incident ultrasonic wave toward an incident direction, each of the reflecting means comprising a triangular-pyramid shaped recess having an apex as a bottom point.
